(19) United States
(12) Patent Application Publication
(10) Pub. No.: US 2014/0322320 A1
(43) Pub. Date: Oct. 30, 2014

(54) NOVEL COMPOSITIONS FOR EMESIS CONTROL IN CANCER PATIENTS UNDERGOING CHEMOTHERAPY AND METHODS THEREOF

(71) Applicant: Uday Saxena, Atlanta, GA (US)

(72) Inventor: Uday Saxena, Atlanta, GA (US)

(21) Appl. No.: 14/260,385

(22) Filed: Apr. 24, 2014

Related U.S. Application Data
(60) Provisional application No. 61/815,340, filed on Apr. 24, 2013.

(51) Int. Cl. A61K 31/7068; A61K 9/28; A61K 9/24; A61K 31/4178

(52) U.S. Cl. CPC A61K 31/7068; A61K 31/4178; A61K 9/2806; A61K 9/209
USPC 424/465; 514/49

(57) ABSTRACT

A novel approach for emesis control in cancer patients undergoing chemotherapy using pharmaceutical formulations comprising a chemotherapeutic agent and anti-emetic agent(s) in combination with different and optimized release profiles is disclosed.

### RELATED APPLICATION

This application is related to and takes priority from U.S. provisional application 61/815,340 filed Apr. 24, 2013 and is herein incorporated in its entirety.

### FIELD OF THE INVENTION

This application relates to a novel formulation comprising a chemotherapeutic agent and anti-emetic agent, each one having a different release profile for treatment of cancer patients undergoing chemotherapy.

### BACKGROUND

Side effects of chemotherapeutic agents, especially the ones that induce acute emesis, have been a concern in treatment of cancer patients. This evidently leads to increased hospitalization time or time spent in outpatient treatment facility, thus leading to increased cost as well as lack of compliance. For example, use of highly emetogenic chemotherapeutic agent such as cisplatin has forced health care provides to identify effective means to deliver the cytotoxic agent but reduce the symptoms associated with such agents. Antiemetic therapy has been keenly worked on to control chemotherapy-induced nausea and vomiting. In this context, serotonin receptor antagonists have shown promise. Previous studies have proposed a combination of 5-hytroxytryptamine-receptor antagonist with a corticosteroid for prevention of nausea and vomiting associated with highly emetic chemotherapy (Annals of Oncology, 1998, 9:811-819). U.S. Pat. No. 5,929,059 provides co-administration of granisetron and dexamethasone for the treatment and/or prophylaxis of nausea and vomiting in cancer patients undergoing chemotherapy and/or radiotherapy. It may be noted that traditionally such antiemetic therapy needs to be administered at least 1 hour prior to the administration of chemotherapy to achieve maximal benefit. A co-formulation of antiemetic agent along with a chemotherapeutic agent with different release profiles has not been observed in the prior art.

### SUMMARY

The present invention envisages co-formulating chemotherapeutic agent with serotonin receptor antagonist as a fixed dose combination wherein the former is provided as regular release or sustained release and the latter is provided as a superior immediate release component or a combination of superior immediate release and sustained release component. Furthermore, a co-formulation of regular release or sustained release chemotherapeutic agent with superior immediate release serotonin receptor antagonist or a combination of superior immediate release and sustained release serotonin receptor antagonist and a corticosteroid is also provided.

The chemotherapeutic agent of the co-formulation is selected from the group consisting of capecitabine, tyrosine kinase inhibitors, platinum complexes and tubulin agents and the anti-emetic agent is 5HT3 receptor antagonist selected from the group consisting of ondansetron, dolasetron, granisetron, palonosetron, ramosetron and tropisetron.

More specifically, the invention provides a co-formulation of therapeutic amounts of capecitabine as a regular release or sustained release agent in combination with ondansetron as a superior immediate release agent or a combination of superior immediate release and sustained release ondansetron for treatment of cancer patients.

The invention also provides a co-formulation of therapeutic amounts of capecitabine as a regular release or sustained release agent in combination with ondansetron as a superior immediate release agent or a combination of immediate release and sustained release ondansetron and optionally dexamethasone for treatment of cancer patients.

In yet another aspect, the pharmaceutical formulation of fixed dose combination wherein the formulation is a film coated tablet comprising an extragranular part comprising an immediate release component and an intragranular part comprising a sustained release component wherein the extragranular part comprises ondansetron hydrochloride dihydrate as the immediate release component, croscarmellose sodium and magnesium stearate and the intragranular part comprises capecitabine as the sustained release component, lactose monhydrate, hydroxy propyl methlyl cellulose and croscarmellose sodium.

The invention also provides a method of treating patients suffering from cancer comprising administering a fixed dose co-formulated combination of anti-emetic agent and chemotherapeutic agent to improve patient compliance and convenience wherein the said agents have different release profiles.

Such formulations of the invention will be beneficial in treatment of cancer patients, especially in increasing compliance as it avoids taking multiple medications at various times of the chemotherapy cycle and as well as reducing cost of administration and greatly improving patient's convenience.

### DESCRIPTION OF THE INVENTION

The present invention relates to co-formulating two different active agents, one which is a cytotoxic agent which essentially treats patients suffering from cancer and the other which is an anti-emetic agent which prevents cytotoxic-agent induced nausea and vomiting in patients undergoing chemotherapy. In addition, the co-formulation may optionally comprise corticosteroid which further prevents acute emesis during cancer therapy. The present invention focuses on co-formulating two different active agents with different release profiles to achieve the desired therapeutic benefit of emesis control with improving patient compliance and convenience coupled with avoidance of multiple drug administrations. For example, the cytotoxic agent of the invention is formulated as a regular release or sustained release component while the anti-emetic agent is co-formulated with the cytotoxic agent as a superior immediate release component or a combination of superior immediate release and sustained release component to prevent the symptoms of nausea and vomiting in patients undergoing cancer chemotherapy.

The unique formulation of the invention provides faster release and effective levels of the anti-emetic agent for prevention of nausea and or vomiting in patients undergoing chemotherapy and permits the simultaneous intake of anti-emetic agent. This strategy of co-formulating two or three active agents avoids the administration of an anti-emetic prior to chemotherapy thus reducing the cost and increasing patient compliance.

The cytotoxic or chemotherapeutic agent of the present invention includes, but is not limited to, capecitabine (Xeloda™, Hoffmann LaRoche), tyrosine kinase inhibitors such as imatinib (Gleevec™, Novartis), platinum complexes such as cisplatin, carboplatin, satraplatin, topoisomerase inihibitors such as etoposide, irinotecan and tubulin agents such as docetaxel, paclitaxel and the likes thereof. Specifically, the cytotoxic agent of the present invention is capecitabine, an orally administered chemotherapeutic agent used for treatment of metastatic breast and colorectal cancers.

The anti-emetic agent of the invention is 5HT3 receptor antagonist which includes, but is not limited to, ondansetron, granisetron, dolasetron, palonosetron, ramosetron or tropisetron. Preferably the anti-emetic agent is ondansetron. By ondansetron reference, the invention includes pharmaceutically acceptable salts, such as hydrochlorides and solvates such as hydrates.

The corticosteroid of the invention is an optional agent which is formulated in conjunction with the chemotherapeutic agent and anti-emetic agent. The preferred corticosteroid is dexamethasone. By dexamethasone, it is understood that the invention includes pharmaceutically acceptable esters thereof.

The formulation of the present invention may be administered by any method known in the art. Some examples of suitable modes of administration include oral or intravenous administration.

In certain embodiments, the present invention is directed to a method of formulating compounds of the present invention in a pharmaceutically acceptable carrier or excipient and may be administered in a wide variety of different dosage forms e.g. tablets, capsules, injectables and the likes thereof. Carriers for injectables may include one or more of solid diluents or fillers, sterile aqueous media, and various nontoxic organic solvents, etc. The present invention provides a co-formulation of multiple active agents with different release profiles. Thus it is a timed release formulation and can be formulated as tablet in tablet which may include compression coated tablets known in the art. Herein pure drug crystals, drug excipient blends, granules, microspheres or beads can be encapsulated inside the core of the dosage form.

The timed release formulation can be multi-layer, two-layer, and/or bilayer tablets for sequential release of two drugs in combination, separate two compatible/incompatible drug substances in which one or both of the drug will be immediately released or one drug is immediate release and other will be sustained/timed release in which one layer is released immediately as initial dose and second layer will be released at required time.

The timed release formulation of the present invention can be capsules containing powder, caplets, and tablet; the formulation herein is hard gelatin/soft gelatin capsules containing one drug in powder form/pellet/microspheres form and other drug will be present as conventional or controlled/timed released formulations. After disintegration of capsules, initially drug from powder/pellet will be released immediately followed by controlled/timed released of other drug present in tablet form.

For oral administration, tablets may contain various excipients, such as one or more of microcrystalline cellulose, sodium citrate, calcium carbonate and the like, along with various disintegrants such as starch and certain complex silicates, together with granulation binders like polyvinylpyrrolidone, sucrose and the like. Solid compositions of a similar type may also be employed as fillers in gelatin capsules.

The formulations of the invention are useful for treatment of cancer patients wherein administration of the anti-cancer agent such as the cytotoxic agents induces emesis in patients. Accordingly, the invention includes cytotoxic agent formulated with 5-HT3 receptor agonist, more specifically capecitabine formulated with ondansetron. The invention provides the formulation in such a manner that the anti-emetic agent is formulated as a superior immediate release component or a combination of superior immediate release and sustained release component and the cytotoxic agent is formulated as a regular release or sustained release component.

Chemotherapeutic agent as used herein includes any anti-cancer agent, cytotoxic agent, anti-proliferative agent which is used for treating patients suffering from any forms of cancer.

By ‘therapeutically effective amount(s)’, it is meant that a dose or dosages recommended by physician that brings about a change for the better in the patient as against a placebo.

Dissolution Profile studies by standard methods known in the art are carried out to test the formulations of the inventions. The co-formulations of the present invention have the following profile:
1. The release of the anti-emetic of the invention is enhanced such that it is superior to the regular immediate release component to achieve rapid and higher blood levels of the anti-emetic agent. For example, 100% dissolution of the active drug substance of the anti-emetic agent is achieved within 30 minutes in the newly developed superior release formulation of the anti-emetic agent (versus the immediate release specifications of 85% dissolution in 60 minutes).
2. A portion of the anti-emetic agent (25 to 75%) is released rapidly (within 30 minutes dissolution of the superior immediate release component) to achieve higher and rapid blood levels of the anti-emetic agent and the remaining portion (75 to 25%) is released in a two hour window to sustain the blood levels of the antiemetic agent to achieve control of the emesis episodes occurring in cancer patients.
3. While the release of the chemotherapeutic agent is comparable to a regular or immediate release component, the added anti-emetic agent conforms to either example 1 or example 2.
4. While the release of the chemotherapeutic agent is sustained over a period of 2-4 hours, the added anti-emetic agent will conform to either example 1 or example 2.
5. The addition of corticosteroid as a regular release component, conforming to the prescribed specifications of immediate release formulations, to the combination of chemotherapeutic agent (examples 3 and 4) and anti-emetic agent (examples 1 and 2) further enhances the anti-emesis control of the co-formulated drug products.

Given below are examples specific to co-formulation combinations of ondansetron as the anti-emetic and capecitabine as the chemotherapeutic agent. The examples provided here are for illustrative purposes and better understanding of the invention and in no way are to be construed as limiting the invention.

### EXAMPLES

### Example 1

### A) Development and Dissolution Kinetics of Ondansetron 2.5 Mg and Capecitabine 500 Mg Film Coated Tablets in Various Formulations (Batch Number 13-691-07)

TABLE 1
Composition of Wet granulation Formulation

| SI | | 13-691-07 |
|---|---|---|
| No. | Ingredients | mg/tab |
| 1 | Capecitabine | 504.32 |
| 2 | Lactose monohydrate | 85.3 |
| | (SuperTab 30GR) | |
| 3 | Hydroxy Propyl Methyl Cellulose | 21.0 |
| | (HPMC 5 cps) | |
| 4 | Croscarmellose sodium (CCS) | 28.0 |
| 5 | Water | qs |
| | Extra granular part | |
| 6 | Ondansetron hydrochloride | 3.122* |
| | dihydrate | |
| 7 | Croscarmellose sodium (CCS) | 40.0 |
| 8 | Magnesium stearate | 5.0 |
| | Total weight of uncoated | 686.75 |
| | tablet | |
| | Film Coating (Opadry pink) | 17.15 |
| | Total weight of film coated | 704 |
| | tablet | |
| | Disintegration time of coated | 8-9 min |
| | tablets 13-691-07 | |
| | Disintegration time of Xeloda | 12-13 min |

*3.122 mg Ondansetron hydrochloride dihydrate is equivalent to 2.5 mg Ondansetron

TABLE 2
Dissolution results of Ondansetron 2.5 mg and Capecitabine
500 mg Film coated tablets(13-691-07) in USP Type II
(Paddle), 50 rpm, 0.1N HCl, 900 ml using HPLC

% Drug release (n = 3)

| Time in min | Ondansetron | Capecitabine |
|---|---|---|
| 5 | 47.3 | 32.0 |
| 10 | 77.6 | 52.0 |
| 15 | 83.9 | 57.2 |
| 20 | 86.8 | 59.1 |
| 30 | 90.5 | 60.6 |
| 45 | 92.7 | 60.1 |
| 200 rpm | 95.1 | 59.1 |
| (Recovery) | | |

TABLE 3
Dissolution results of Ondansetron 2.5 mg and Capecitabine
500 mg Film coated tablets (13-691-07) in USP Type
II (Paddle), 50 rpm, Water, 900 ml using HPLC

% Drug release (n = 3)

| Time in min | Ondansetron | Capecitabine |
|---|---|---|
| 5 | 2.8 | 25.1 |
| 10 | 9.3 | 66.0 |
| 15 | 13.7 | 83.9 |
| 20 | 16.9 | 92.7 |
| 30 | 19.7 | 98.9 |
| 45 | 21.2 | 100.8 |
| 200 rpm | 23.1 | 100.6 |
| (Recovery) | | |

### Observations:

1. As provided in Table 1, Batch No. 13-691-07 was formulated by wet granulation process. The objective was to achieve quicker release of Ondansetron followed by Capecitabine release. Intra granular part contains Capecitabine 500 mg with lactose as filler, HPMC (in water) as binder, Croscarmellose sodium (CCS) as disintegrant. The extra granular part contains Ondansetron drug with CCS as disintegrant for quicker release of Ondansetron and Magnesium stearate as a lubricant.
2. The dissolution of Batch No. 13-691-07 was performed in two different dissolution media i.e. and 0.1N HCl (Table 2) and water (Table 3). The dissolution was analyzed using HPLC.
3. The dissolution profile of Ondansetron in 0.1N HCl was found to be 77.6% (n=3). However, as it can be seen from the attached individual tablet data, one of the tablets showed lower release even at 45 min, probably because of lower drug content. The batch size was very small which could have caused content homogeneity issue. Improved homogeneity can be expected from a larger batch and testing of 6 tablets will give additional data on tablet to tablet variability.
4. Capecitabine dissolution in water was found to be excellent—reaching 100% in 45 minutes. Whereas dissolution in 0.1 N HCl was found to reach only up to 60% in 45 min (Table 3). This finding is supported by literature data suggesting possible degradation of Capecitabine in acidic medium.
5. From the results discussed above, water for capecitabine and 0.1N HCl for ondansetron are found to be appropriate dissolution medium for the formulation of the invention.

### B) Comparative Dissolution Profile of Prototype Tablets (Ondansetron with Capecitabine) and Xeloda Tablets in Different Dissolution Media

In continuation with the previous observations, further dissolution studies on prototype formulation was carried out with Xeloda tablets to compare the drug release profile and the observations are described below.

### I. Dissolution of Tablets in Water as Dissolution Medium

TABLE 4
Dissolution results of Capecitabine in Xeloda
(X2755B03) & Batch No. 13-691-07 Tablets, USP
Type II (Paddle), 50 rpm, 900 ml using HPLC

| | Xeloda (X2755B03) | Prototype 13-691-07 |
|---|---|---|
| | (n = 3) | (n = 6) |
| | Water | Water |
| | % Release of | % Release of |
| Time (Min) | Capecitabine | Capecitabine |
| 5 | 27.7 | 26.2 |
| 10 | 57.4 | 67.8 |
| 15 | 75.9 | 87.0 |
| 20 | 90.1 | 94.5 |
| 30 | 99.2 | 99.8 |
| 45 | 100.4 | 101.1 |
| 200 rpm | 100.7 | 100.8 |

### Observation:

Dissolution study of XELODA tablets (n=3) and the co-formulated tablets (Batch No. 13-691-07) (n=6) of the invention were carried out in water as dissolution media to compare release of Capecitabine. Dissolution results of Xeloda tablets were found to be closely matching with 13-691-07 in water.

### II) Dissolution of Tablets in HCl as Medium

Studies have suggested the degradation of capecitabine in 0.1N HCl as dissolution medium. To assess the impact of 0.1N HCl on the release profile, dissolution study was carried out only on prototype tablets (13-691-07). The dissolution results confirmed the impact on the degradation of Capecitabine and only 60% drug was recovered after 45 min. Hence dissolution study of Xeloda was not performed in 0.1 N HCl.

TABLE 5
Dissolution results of Capecitabine
using 0.1N HCl as Dissolution Medium

| | 13-691-07 (n = 3) |
|---|---|
| | 0.1N HCl |
| | % Release of |
| Time(Min) | Capecitabine |
| 5 | 32 |
| 10 | 52 |
| 15 | 57.2 |
| 20 | 59.1 |
| 30 | 60.6 |
| 45 | 60.1 |
| 200 rpm | 59.1 |

Studies also suggest usage of diluted acidic media (0.01N HCl) suitable for testing Capecitabine tablets. Hence, further dissolution study of Xeloda and 13-691-07 were performed with 0.01N HCl (pH 2). The results (Table 6) suggested slightly faster release of prototype tablets than Xeloda tablets.

TABLE 6
Dissolution result of Xeloda and 13-691-07 in 0.01N HCl

| | Xeloda Tablets (n = 3) | 13-691-07 (n = 3) |
|---|---|---|
| | 0.01N HCl | 0.01N HCl |
| | % Release of | % Release of |
| Time(Min) | Capecitabine | Capecitabine |
| 5 | 18.7 | 27.1 |
| 10 | 37.1 | 57.6 |
| 15 | 48.8 | 69.6 |
| 20 | 65.4 | 79.6 |
| 30 | 80.4 | 89.4 |
| 45 | 87.3 | 93.2 |
| 200 rpm | 91.9 | 92.7 |

### III) Dissolution Profile of Ondansetron

Pharmacopoeial monographs and OGD specify different dissolution media for Ondansetron A) Water or B) 0.1N HCl.

For Capecitabine, water is recommended as dissolution media due to its instability in acidic medium. Dissolution study of 13-691-07 was carried out in 0.1N and 0.01N HCl for Ondansetron. The dissolution results suggested that the release of Ondansetron is dependent on the acidic concentration, releasing more in 0.1N HCl compared to 0.01N.

TABLE 7
Dissolution profile of Ondansetron in 0.1N and 0.01N HCl

| | 13-691-07 (n = 3) | 13-691-07 (n = 3) |
|---|---|---|
| | 0.1N HCl | 0.01N HCl |
| | % Release of | % Release of |
| Time(Min) | Ondansetron | Ondansetron |
| 5 | 47.3 | 30.5 |
| 10 | 77.6 | 63.3 |
| 15 | 83.9 | 73.7 |
| 20 | 86.8 | 83.3 |
| 30 | 90.5 | 92.5 |
| 45 | 92.7 | 96.3 |
| 200 rpm | 95.1 | 96.7 |

Based on the results of dissolution studies for both Capecitabine and Ondansetron, 0.01N HCl appears to be preferred dissolution medium for both molecules.

### Example 2

### Development and Dissolution Kinetics of Ondansetron 2.5 Mg and Capecitabine 500 Mg Film Coated Tablets in Various Formulations (Batch Number 13-691-08)

TABLE 8
Composition of Wet granulation Formulation

| SI | | 13-691-08 |
|---|---|---|
| No. | Ingredients | mg/tab |
| 1 | Capecitabine | 504.32 |
| 2 | Lactose monohydrate | 85.3 |
| | (SuperTab 11SD) | |
| 3 | Hydroxy Propyl Methyl Cellulose | 21.0 |
| | (HPMC 5 cps) | |
| 4 | Croscarmellose sodium (CCS) | 20.0 |
| 5 | Water | qs |
| | Extra granular part: | |
| 6 | Ondansetron hydrochloride | 3.122* |
| | dihydrate | |
| 7 | Croscarmellose sodium (CCS) | 45.26 |
| 8 | Lactose monohydrate | 50.00 |
| | (SuperTab 11SD) | |
| 9 | Magnesium stearate | 6.0 |
| | Total weight of uncoated | 735 |
| | tablet | |
| | Film Coating (Opadry pink) | 15 |
| | Total weight of film coated | 750 |
| | tablet | |
| | Disintegration time of coated | 6-7 min |
| | tablets 13-691-08 | |
| | Disintegration time of Xeloda | 12-13 min |

*3.122 mg Ondansetron hydrochloride dihydrate is equivalent to 2.5 mg Ondansetron

TABLE 9
Dissolution results of Ondansetron 2.5 mg and Capecitabine
500 mg Film coated tablets in USP Type II (Paddle),
50 rpm, 0.01N HCl, 900 ml using HPLC

| | % Drug release in 0.01N HCl | | | | |
|---|---|---|---|---|---|
| | Ondansetron | | Capecitabine | | |
| | 13-691-07 | 13-691-08 | XELODA | 13-691-07 | 13-691-08 |
| Time in min | (n = 3) | (n = 6) | (n = 3) | (n = 3) | (n = 6) |
| 5 | 30.5 | 36.3 | 18.7 | 27.1 | 29.7 |
| 10 | 63.3 | 70.4 | 37.1 | 57.6 | 58 |
| 15 | 73.7 | 88.5 | 48.8 | 69.6 | 74.4 |
| 20 | 83.3 | 96.8 | 65.4 | 79.6 | 81.8 |
| 30 | 92.5 | 104.8 | 80.4 | 89.4 | 88.3 |
| 45 | 96.3 | 109.3 | 87.3 | 93.2 | 91.6 |
| 200 rpm | 96.7 | 110 | 91.9 | 92.7 | 91 |
| (Recovery) | | | | | |

### Observation:

1. Batch No. 13-691-08 was formulated with increased tablet weight from 700 mg to 750 mg by adding Lactose and increasing Croscarmellose sodium in extra granular part to improve release profile of Ondansetron in 0.01N HCl.
2. The initial dissolution profile of current batch 13-691-08 is slightly faster compared to previous batch 13-691-07 at the 5^{th} minute and same is continued till the 45^{th} minute. The increased release profile might be attributed to two tablets with higher API concentration—which might be due to non-homogeneity.
3. At the end of 15^{th} minute this current batch is releasing the Ondansetron about 88% which is more than the target profile of 80%.
4. The dissolution profile of Capecitabine in 0.01N HCl from batch no. 13-691-08 was found to be slightly higher than XELODA.

### Example 3

### Development and Dissolution Kinetics of Ondansetron 2.5 Mg and Capecitabine 500 Mg Film Coated Tablets in Various Formulations (Batch Number 13-691-09 and 13-691-11) and Formulation Trials with Ondansetron Micronized API

Based on the previous trial results, micronized form of Ondansetron API was used. The micronized API was found to be fluffy and sticking to surfaces, which could be due to increased surface area.

The details of the formulations (13-691-09 and 13-691-11) executed using the micronized API are summarized in the below Table 10. 13-691-08 was executed with regular grade of API.

TABLE 10
Formulations using the micronized API

| SI | | 13-691-08 | 13-691-09 | 13-691-11 |
|---|---|---|---|---|
| No. | Ingredients | mg/tab | mg/tab | mg/tab |
| 1 | Capecitabine | 504.32 | 504.32 | 504.32 |
| 2 | Lactose monohydrate | 85.3 | 85.3 | 86.3 |
| | (SuperTab 11SD) | | | |
| 3 | Hydroxy Propyl Methyl | 21.0 | 21.0 | 21.0 |
| | Cellulose (HPMC 5 cps) | | | |
| 4 | Croscarmellose sodium (CCS) | 20.0 | 20.0 | 20.0 |
| | Extra granular part: | | | |
| 5 | Ondansetron hydrochloride | 3.122* | — | — |
| | dihydrate, d(0.9) = 304 μm | | | |
| 6 | Ondansetron hydrochloride | — | 3.120* | 3.120* |
| | dihydrate, d(0.9) = 10.78 μm | | | |
| 7 | Croscarmellose sodium (CCS) | 45.26 | 45.26 | 45.26 |
| 8 | Lactose monohydrate | 50.00 | 50.00 | 50.00 |
| | (SuperTab 11SD) | | | |
| 9 | Magnesium stearate | 6.0 | 6.0 | 5.0 |
| | Total weight of uncoated | 735 | 735 | 735 |
| | tablet | | | |
| | Film Coating (Opadry pink) | 15 | 15 | 15 |
| | Total weight of film coated | 750 | 750 | 750 |
| | tablet | | | |

*3.120-3.122 mg Ondansetron hydrochloride dihydrate is equivalent to 2.5 mg Ondansetron

TABLE 11
Content uniformity of Ondansetron 2.5 mg and Capecitabine 500 mg
Content uniformity of Ondansetron 2.5 mg and Capecitabine 500 mg

| Tablet | | | | |
|---|---|---|---|---|
| no./Batch | 13-639-08 | | 13-639-09 | |
| no. | Ondansetron | Capecitabine | Ondansetron | Capecitabine |
| 1 | 100.8 | 102.1 | 80.8 | 94.0 |
| 2 | 101.2 | 103.6 | 88.0 | 102.6 |
| 3 | 98.4 | 102.9 | 90.0 | 102.7 |
| 4 | 102.4 | 102.6 | 92.8 | 102.1 |
| 5 | 103.6 | 103.2 | 90.8 | 103.7 |
| Average | 101.3 | 102.9 | 88.5 | 101.0 |
| SD | 1.95 | 0.57 | 4.63 | 3.97 |
| RSD | 1.9 | 0.6 | 5.2 | 3.9 |
| (M-X) | −0.20 | 1.40 | −13.00 | −0.50 |
| K | 2.4 | 2.4 | 2.4 | 2.4 |
| kS | 4.68 | 1.37 | 11.11 | 9.53 |
| Acceptance | 4.5 | 2.8 | −1.9 | 9.0 |
| value (L1) | | | | |

TABLE 12
Comparative dissolution results of Ondansetron 2.5 mg and Capecitabine 500 mg Film
coated tablets in USP Type II (Paddle), 50 rpm, 0.01N HCl, 900 ml using HPLC

| | % Drug release in 0.01N HCl | | | | | | |
|---|---|---|---|---|---|---|---|
| | Ondansetron | | | Capecitabine | | | |
| Time in | 13-691-08 | 13-691-09 | 13-691-11 | XELODA | 13-691-08 | 13-691-09 | 13-691-11 |
| min | (n = 6) | (n = 6) | (n = 6) | (n = 3) | (n = 6) | (n = 6) | (n = 6) |
| 5 | 36.9 | 27.3 | 18.5 | 18.7 | 34.5 | 29.8 | 17.9 |
| 10 | 70.9 | 54.1 | 44.7 | 37.1 | 67.7 | 58.0 | 45.2 |
| 15 | 89.2 | 72.3 | 66.3 | 48.8 | 83.5 | 76.4 | 66.8 |
| 20 | 97.9 | 79.3 | 79.1 | 65.4 | 91.5 | 83.8 | 79.8 |
| 30 | 101.7 | 83.8 | 87.7 | 80.4 | 95.4 | 87.8 | 89.9 |
| 45 | 103.2 | 85.3 | 92.2 | 87.3 | 95.8 | 89.0 | 94.7 |
| 200 rpm | 103.6 | 85.3 | 93.5 | 91.9 | 95.2 | 88.8 | 96.7 |
| (Recovery) | | | | | | | |

### Observations:

Using the batch containing micronized ondansetron API (Batch No 13-691-09), the results of the dissolution rate were slower compared to earlier formulation processed with regular grade API-13-691-08. The rate of dissolution was found to be less and even the extent of dissolution was less at the end of 45 minutes (85.3% release at the end of 45 minutes). The reason for lower rate and extent of dissolution might be attributed to the challenge to handle API with static charges which might have led to API loss.
The variation in the % release between tablets at different time points had improved with lesser RSD values (13-691-09). As per the earlier assumptions, the micronized form of API is distributing well in the blend thereby yielding the uniform dissolution results at all-time points.
An additional batch was processed with minor changes (Batch No. 13-691-11). The dissolution results of this batch were better compared to earlier batch #09. The rate of the dissolution did not improve, but the extent of dissolution at the end of 45^{th} minute improved.
The individual tablets dissolution results of 13-691-11 are acceptable with less than 10% RSD at all-time points which confirms the uniform distribution and release of micronized API.

### CONCLUSION

Based on the formulation trials conducted the following is concluded:
The grade of Ondansetron API required to process formulation has been identified—Micronized form with a target particle size. The micronized form of API will uniformly distribute in the formulation thereby resulting in uniform dissolution results. Furthermore, the studies provide a pharmaceutical co-formulation of fixed dose combination of capecitabine and ondansetron wherein the formulation comprises capecitabine as a regular release or sustained release component and ondansetron as a superior immediate release or a combination of superior immediate release and sustained release component.

I claim:

1. A pharmaceutical co-formulation of fixed dose combination, comprising therapeutically effective amounts of chemotherapeutic agent and anti-emetic agent, wherein the co-formulation comprises the chemotherapeutic agent as a regular release or sustained release component and the anti-emetic agent as a superior immediate release component or a combination of superior immediate release and sustained release component.

2. A pharmaceutical co-formulation of fixed dose combination, comprising therapeutically effective amounts of chemotherapeutic agent and anti-emetic agent and optionally a corticosteroid, wherein the co-formulation comprises the chemotherapeutic agent as a regular release or sustained release component and the anti-emetic agent as a superior immediate release component or a combination of superior immediate release and sustained release component.

3. The pharmaceutical co-formulation of fixed dose combination of claim 1, wherein the chemotherapeutic agent is selected from the group consisting of capecitabine, tyrosine kinase inhibitors, platinum complexes and tubulin agents and the anti-emetic agent is 5HT3 receptor antagonist selected from the group consisting of ondansetron, dolasetron, granisetron, palonosetron, ramosetron and tropisetron.

4. The pharmaceutical co-formulation of fixed dose combination of claim 2, wherein the chemotherapeutic agent is selected from the group consisting of capecitabine, tyrosine kinase inhibitors, platinum complexes and tubulin agents and the anti-emetic agent is 5HT3 receptor antagonist selected from the group consisting of ondansetron, dolasetron, granisetron, palonosetron, ramosetron and tropisetron.

5. A pharmaceutical co-formulation of fixed dose combination comprising therapeutically effective amounts of capecitabine and ondansetron, wherein the formulation comprises capecitabine as a regular release or sustained release component and ondansetron as a superior immediate release or a combination of superior immediate release and sustained release component.

6. A pharmaceutical co-formulation of fixed dose combination comprising therapeutically effective amounts of capecitabine, ondansetron and optionally dexamethosone, wherein the formulation comprises capecitabine as a regular release or sustained release component and ondansetron as a superior immediate release component or a combination of superior immediate release and sustained release component.

7. A pharmaceutical co-formulation of fixed dose combination wherein the formulation is a film coated tablet comprising an extragranular part comprising an immediate release component and an intragranular part comprising a sustained release component wherein the extragranular part comprises ondansetron hydrochloride dihydrate as the immediate release component, croscarmellose sodium and magnesium stearate and the intragranular part comprises capecitabine as the sustained release component, lactose monhydrate, hydroxy propyl methlyl cellulose and croscarmellose sodium.

8. A method of treating patients suffering from cancer comprising administering a fixed dose co-formulated combination of anti-emetic agent and chemotherapeutic agent to improve patient compliance and convenience wherein the said agents have different release profiles.
